(19) United States
(12) Patent Application Publication
(10) Pub. No.: US 2025/0270254 A1
(43) Pub. Date: Aug. 28, 2025

(54) PROBES AND KITS FOR THE EARLY DIAGNOSIS OF DIFUSE LARGE B-CELL LYMPHOMA

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Zijian ZHAO, Beijing (CN); Ding DING, Beijing (CN); Siyi YIN, Beijing (CN)

(21) Appl. No.: 18/246,237

(22) PCT Filed: Jun. 29, 2022
(86) PCT No.: PCT/CN2022/102326

§ 371 Date: Mar. 22, 2023

(51) Int. Cl. C07K 7/06; C07K 7/08; C07K 17/14; G01N 33/574; G01N 33/58

(52) U.S. Cl. CPC C07K 7/06; C07K 7/08; C07K 17/14; G01N 33/57492; G01N 33/582; G01N 2800/7028

(57) ABSTRACT

The present disclosure provides a probe specifically binding to CD138, a kit and a microfluidic chip comprising the probe, and a method of diagnosing diffuse large B-cell lymphoma in a subject using the probe, the kit, or the microfluidic chip. The present disclosure also provides a method of screening the probe for diagnosing diffuse large B-cell lymphoma.

### FIELD OF THE INVENTION

The present disclosure relates to the field of biomedical technology, specifically to a probe specifically binding to CD138, a kit and a microfluidic chip comprising the probe, and a method for diagnosing diffuse large B-cell lymphoma using the probe, the kit, or the microfluidic chip, and also to a method of screening the probe for diagnosing diffuse large B-cell lymphoma.

### TECHNICAL BACKGROUND

Common hematologic tumors mainly include various types of leukemia, multiple myeloma, and malignant lymphoma, among which the malignant lymphoma has a very high malignancy degree and fatality rate, and has become one of the major research hotspots in the medical community in recent years. Diffuse large B-cell lymphoma is the most common type of non-Hodgkin's lymphoma (NHL), accounting for almost ⅓ of all cases. This lymphoma accounts for the majority of cases of previous clinically “aggressive” or “moderately to highly malignant” lymphomas. The proper diagnosis of diffuse large B-cell lymphoma requires hematopathologists based on appropriate biopsy and the evidence of B-cell immunophenotyping. In recent years, research data from several international multicenter randomized controlled clinical trials have demonstrated that the standard first-line treatment regimen for this lymphoma should be Rituximab (R)+CHOP regimen. And better efficacy is achieved by increasing the dose density of the regimen and shortening the time between courses, as in the case of R-CHOP14 regimen. However, due to the difficulty of sampling for pathologic analysis and the occurrence of pathologic analysis often in the middle and advanced stages from disease onset, patients usually do not receive effective and timely treatment, which explains the high mortality of diffuse large B-cell lymphoma. Recent studies have demonstrated that CD138 shows varying degrees of expression with lymphocyte development. CD138 is highly expressed on the surface of tumor cells, particularly circulating tumor cells, in patients with diffuse large B-cell lymphoma. However, due to limitations in detection sensitivity, there is still no effective method for early screening of diffuse large B-cell lymphoma to date. Therefore, the design of high-affinity probes and highly sensitive detection methods and reagents for diffuse large B-cell lymphoma has become a research hotspot in scientific and medical communities.

Polypeptide probes have the characteristics of good selectivity, low immunogenicity, good biocompatibility, strong penetration and easy clearance by excretion, etc. Such polypeptide probes show strong superiority in cancer diagnosis, and even have the trend to replace traditional antibody-based diagnosis and treatment reagents. However, the preparation and screening process of conventional probe is highly randomized and usually requires extensive verification experiments to complete the screening one by one, resulting in a long-time period and high cost of preparation for polypeptide probes.

### SUMMARY OF THE INVENTION

To address the above technical problems, the present disclosure provides a probe specifically binding to CD138, a kit and a microfluidic chip comprising the probe, and a method of diagnosing diffuse large B-cell lymphoma in a subject using the probe, the kit, or the microfluidic chip. The present disclosure also provides a method of screening the probe for diagnosing diffuse large B-cell lymphoma.

High-affinity probes can be obtained quickly and effectively by the screening design scheme for preparing the polypeptide probe of the present disclosure. Specifically, in the method of screening the probe of the present disclosure, the diversity of molecular probes can be effectively improved by the construction of the library of polypeptide probes by the Mix-Split method, and the screening of effective probes can be further conducted by high-throughput screening methods, thereby solving the problem of detection sensitivity in the early diagnosis of cancers and saving time and cost. The present disclosure provides a kit and a microfluidic chip with the advantages of high throughput, fast analysis speed, low contamination, small amount of samples required, inexpensiveness and safety, etc., which have broad development prospects in the field of clinical diagnosis and disease screening. The combination of high-affinity small molecular probes with microfluidic chips can effectively improve the sensitivity and timeliness of detection, while further reducing the complexity and cost of operation, which is the future trend of diagnosis and a new strategy for the early diagnosis of cancers.

Accordingly, in one aspect, the present disclosure provides a probe specifically binding to CD138 having, from the N-terminus to the C-terminus, the following structure:

X-M-Arg-Y-Phe or X-M-Arg-Y-Ile,

wherein X is any amino acid residue, Mis any amino acid residue or is absent, and the number of amino acid residues represented by X+M ranges from 3-15, e.g., 3, 4, 5, 6, 7, 8, 9, 10, 11, 12, 13, 14, or 15, and the N-terminal amino acid residues or the amino acid residues represented by X+M are overall hydrophilic, and wherein Y is selected from one or more of the following amino acid residues: Arg, Gly, Tyr, Asn, Gln, Ser, Thr, Cys, and Sec, and the number of amino acid residues represented by Y ranges from 1-12, e.g., 1, 2, 3, 4, 5, 6, 7, 8, 9, 10, 11, or 12, and preferably the probe has a length ranging from 5-18 amino acids, e.g., a length of 5, 6, 7, 8, 9, 10, 11, 12, 13, 14, 15, 16, 17 or 18 amino acids.

In some embodiments of the probe of the present disclosure, the N-terminal amino acid residues or the amino acid residues represented by X+M, and/or the C-terminal amino acid residues or the amino acid residues represented by Arg-Y-Phe or Arg-Y-Ile have a π-πstacking capability.

In some preferred embodiments, the probe is selected from the group consisting of:

| | Bta893: |
|---|---|
| (SEQ ID NO: 1) | |
| | His-Cys-Trp-Arg-Gly-Phe; |
| | Bta1335: |
| (SEQ ID NO: 2) | |
| | Cys-Cys-His-Arg-Gly-Phe; |
| | Bta3097: |
| (SEQ ID NO: 3) | |
| | Gly-Cys-Tyr-Arg-Arg-Phe; |
| | BtaP1: |
| (SEQ ID NO: 4) | |
| | His-Cys-Trp-Arg-Arg-Phe; |
| | BtaP2: |
| (SEQ ID NO: 5) | |
| | Ile-Cys-Trp-Arg-Arg-Phe; |
| | and |
| | BtaP3: |
| (SEQ ID NO: 6) | |
| | Ile-Cys-Trp-Arg-Arg-Gly-Ile. |

In some embodiments, the X is Ile-Cys-Trp.

In some preferred embodiments, the probes are selected from the group consisting of:

| | BtaPL1: |
|---|---|
| (SEQ ID NO: 7) | |
| | Ile-Cys-Trp-Arg5-Phe; |
| | BtaPL2: |
| (SEQ ID NO: 8) | |
| | Ile-Cys-Trp-Arg7-Phe; |
| | BtaPL3: |
| (SEQ ID NO: 9) | |
| | Ile-Cys-Trp-Arg9-Phe; |
| | BtaPL4: |
| (SEQ ID NO: 10) | |
| | Ile-Cys-Trp-Arg11-Phe; |
| | and |
| | BtaPL5: |
| (SEQ ID NO: 11) | |
| | Ile-Cys-Trp-Arg13-Phe. |

In some embodiments, the Y is selected from one or both of the following amino acid residues: Arg and Gly.

In some preferred embodiments, the probe is selected from the group consisting of:

| | BtaE1: |
|---|---|
| (SEQ ID NO: 12) | |
| | Ile-Cys-Trp-Arg3Gly3-Phe; |
| | BtaE2: |
| (SEQ ID NO: 13) | |
| | Ile-Cys-Trp-Gly2Arg3Gly3-Phe; |
| | and |
| | BtaE3: |
| (SEQ ID NO: 14) | |
| | Ile-Cys-Trp-Gly-Arg3-Gly2-Phe. |

In another aspect, the present disclosure provides a kit comprising the probe of the present disclosure.

In some embodiments of the kit of the present disclosure, the probe is labeled by fluorescein or biotin, for example by Rhodamine B.

In some embodiments, the kit further comprises a surfactant, a buffer, and EDTA, preferably wherein the surfactant is selected from one or a combination of Tween 20, sodium dodecyl sulfate (SDS), and sorbitan fatty acid esters (Span), and preferably wherein the buffer is PBS. In some preferred embodiments, the molar concentration of the Rhodamine B-labeled probe in the kit is 0.5 mmol/L to 50 mmol/L, e.g., 2-40 mmol/L, 5-35 mmol/L, 10-30 mmol/L, 15-25 mmol/L.

In some preferred embodiments, the volume percentage of the surfactant in the kit is 0.5%-5%, preferably 1%; the pH value of the buffer system is 7.4; the molar concentration of EDTA is 0.5 mmol/L to 50 mmol/L, e.g. 2-40 mmol/L, 5-35 mmol/L, 10-30 mmol/L, 15-25 mmol/L.

In yet another aspect, the present disclosure provides a microfluidic chip comprising the probe of the present disclosure immobilized on a solid substrate. In some embodiments of the microfluidic chip of the present disclosure, the probe is coupled to the surface of the solid substrate. In some embodiments, the solid substrate is glass.

In another aspect, the present disclosure provides a method of screening a probe for diagnosing diffuse large B-cell lymphoma, for example, a method for obtaining the probe of the present disclosure, preferably comprising the steps of:
i) constructing a probe library, e.g., constructing a high-throughput library of probes by using a Mix-Split method,
ii) enriching the probe by using CD138, e.g., performing enrichment and preliminary screening using magnetic beads,
iii) optimizing the probes, e.g., performing high-throughput screening using a surface plasmon resonance imaging technology, for the following features:
a) the probe having, from the N-terminus to the C-terminus, the following structure: X-M-Arg-Y-Phe or X-M-Arg-Y-Ile, wherein X is any amino acid residue, M is any amino acid residue or absent, and the number of N-terminal amino acid residues or amino acid residues represented by X+M ranges from 3-15, e.g., 3, 4,5, 6, 7, 8, 9, 10, 11, 12 13, 14 or 15,
b) the N-terminal amino acid residues or the amino acid residues represented by X+M being overall hydrophilic,
c) Y being selected from one or more of the following amino acid residues: Arg, Gly, Tyr, Asn, Gln, Ser, Thr, Cys, and Sec, and the number of amino acid residues represented by Y ranging from 1-12, e.g., 1, 2, 3, 4, 5, 6, 7, 8, 9, 10, 11 or 12, and preferably
d) the probe having a length ranging from 5-18 amino acids, e.g., a length of 5, 6, 7, 8, 9, 10, 11, 12, 13, 14, 15, 16, 17 or 18 amino acids.

In some embodiments of the method of the present disclosure, the method further comprises optimizing the probe for the feature selected from:
e) the N-terminal amino acid residues or amino acid residues represented by X+M, and/or the C-terminal amino acid residues or amino acid residues represented by Arg-Y-Phe or Arg-Y-Ile having a π-π stacking ability,
f) the X being Ile-Cys-Trp, and
g) the Y being selected from one or both of the following amino acid residues: Arg and Gly.

In yet another aspect, the present disclosure provides a method of diagnosing diffuse large B-cell lymphoma in a subject, comprising
a) obtaining a blood sample from the subject,
b) obtaining a mononuclear cell fraction from the blood sample,
c) contacting the probe of the present disclosure, the kit of the present disclosure, the microfluidic chip of the present disclosure, or the probe obtained by screening using the method of the present disclosure with the mononuclear cell fraction, and
d) detecting whether lymphoma cells are captured using visualization means, thereby diagnosing whether the subject suffers from diffuse large B-cell lymphoma.

In some embodiments, the present disclosure provides the probe of the present disclosure, the kit of the present disclosure, the microfluidic chip of the present disclosure, or the probe obtained by screening using the method of the present disclosure, for use in diagnosing diffuse large B-cell lymphoma in a subject.

In some embodiments, the present disclosure provides use of the probe of the present disclosure, the kit of the present disclosure, the microfluidic chip of the present disclosure, or the probe obtained by screening using the method of the present disclosure in the preparation of a composition for diagnosing diffuse large B-cell lymphoma in a subject.

The present invention has the following beneficial effects compared with the prior technology:
(1) The technology provided in the present disclosure enables early diagnosis of diffuse large B-cell lymphoma with ease of operation and high sensitivity, compared to traditional technologies;
(2) The kit and the coupling manner of microfluidic chip structures with probes provided in the present disclosure have the ability of large-scale mass production and is universal for various types of diseases;
(3) The design and screening method of the probe provided in the present disclosure is faster and more economical than traditional methods and design ideas, and has the possibility of large-scale promotion, providing new strategies and ideas for diagnosis.

### Description of the Drawings

An understanding of the features and advantages of the present invention may be obtained by reference to the following detailed description and accompanying drawings describing exemplary embodiments utilizing the principles of the present invention, in the accompanying drawings:

FIG. 1 is a schematic diagram of the basic principle of preliminary screening of probes.

FIG. 2 shows the results of the detection of probes Bta893, Bta1335 and Bta3097 using CD138 by the surface plasmon resonance imaging technology.

FIG. 3 shows the results of the detection of probes BtaP1, BtaP2 and BtaP3using CD138 by the surface plasmon resonance imaging technology.

FIG. 4 shows the results of the detection of probes BtaPL1, BtaPL2, BtaPL3,BtaPL4 and BtaPL5 using CD138 by the surface plasmon resonance imaging technology.

FIG. 5 shows the results of the detection of probes BtaE1, BtaE2 and BtaE3using CD138 by the surface plasmon resonance imaging technology.

FIG. 6 shows the characterization results of flow cytometry.

FIG. 7 is a schematic diagram of the technological process of microfluidic chips.

FIG. 8 shows the results of the detection of diffuse large B-cell lymphoma cells using probes.

### EXAMPLES

The present invention is further illustrated by the following examples, but any example or combination thereof should not be construed as the limitation of the scope or embodiments of the present invention. The scope of the present invention is defined by the appended claims, and the scope defined by the claims can be clearly understood by those of ordinary skill in the art, in conjunction with the specification and general knowledge in the art. Those skilled in the art may make any modification or alteration to the technical solution of the present invention without deviating from the spirit and scope of the present invention, and such modification and alteration is also included within the scope of the present invention.

The experimental methods used in the following examples, if not otherwise specified, are conventional methods.

The materials, reagents and the like used in the following examples, if not otherwise specified, are available from commercial sources.

Unless otherwise specified, the “nM” herein refers to “n mol/L”, the “μM” refers to “μ mol/L”, and the “mM” refers to “m mol/L”.

### Example 1. Synthesis, Screening and Optimization of Probe Libraries

### 1. Synthesis and Preliminary Screening of Polypeptide Probe Libraries

The polypeptide probe library was constructed using the Mix-Split method and the specific steps were as follows:
1) 150 mg of Tentagel-NH₂ resin was weighed, and subjected to cycles according to the solid-phase polypeptide synthesis procedure (the resin was immersed in 10% hexahydropyridine (DMF) solution to deprotect and fully swell; the resin was washed with DMF three times prior to the next reaction step; amino acids were mixed with HBTU 1:1 and then added to the reaction tube loaded with the resin for reaction; after completion, the hexahydropyridine was added to deprotect for the next round), in which 180 mg of His, Gly, and Cys were added in sequence to react in sequence for three cycles;
2) After the reaction was completed, the resin was divided equally into 3 parts and 60 mg of Cys, Leu and Arg were added respectively to each tube for coupling with an equal amount of HBTU, and after the coupling was completed, the 3 tubes of resin were mixed and deprotected;
3) The resin was again divided equally into 3 parts, and 60 mg of Trp, Leu and Asn were added respectively to each tube for coupling with an equal amount of HBTU, and after the coupling was completed, the 3 tubes of resin were mixed and deprotected;
4) The resin was again divided equally into 3 parts, and 45 mg of Arg, Trp and Phe were added respectively to each tube for coupling with an equal amount of HBTU, and after the coupling was completed, the 3 tubes of resin were mixed and deprotected;
5) The resin was again divided equally into 5 parts, and 36 mg of Gly, Phe, Leu, Asp and Ser were added respectively to each tube for coupling with an equal amount of HBTU, and after the coupling was completed, the 5 tubes of resin were mixed and deprotected;
6) The resin was again divided equally into 5 parts, and 36 mg of Phe, Ser, Leu, Asp, and Tyr were added respectively to each tube for coupling with an equal amount of HBTU, and after the coupling was completed, the 5 tubes of resin were mixed and deprotected;
7) After the above steps, solvent replacement and resin shrinkage were performed using methanol, and a dried resin loaded with a polypeptide library was obtained following vacuum dry;
8) The peptide chain was then cleaved from the resin under strong acids while the side chain protecting group was removed to obtain the probe;
9) CD138 was connected to magnetic beads modified with carboxyl groups at the end using EDC: NHS 1:1, and the modified beads were mixed and interacted with the probe library for 30 min after sufficiently washed with PBS, and the bound probes were enriched down using a magnetic field to obtain the preliminarily screened affinity-bound probes.

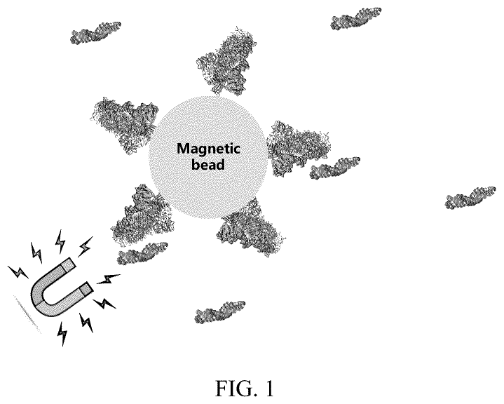

The schematic diagram of the preliminary screening of probes is shown in FIG. 1. Next, the alternative probes are further screened using the surface plasmon resonance imaging technology.

### 2. Screening of Probes

High-throughput screening of the synthetic probes was performed using the surface plasmon resonance imaging technology and the specific steps were as follows:
1) The synthesized probe libraries were dissolved into ddH₂O to obtain probe samples at a concentration of 100 μg/mL, respectively;
2) The probe samples were spotted on the surface of a bare gold chip, with three replicates for each sample, incubated at 4° C. for 12 hours and then the chip was immersed in 5% skimmed milk and blocked at 4° C. for 12 hours, washed with 10X PBS, 1 XPBS and ultrapure water in sequence, and blow-dried by nitrogen gas;
3) The chip was mounted on the SPRi instrument, and the SPRi angle was assayed and adjusted to the optimal optical position, the relevant detection points including sample points and blank points were selected in the detection area, and the experimental flow rate was set to 2 μL/s;
4) PBS was selected as the buffer to pass into the flow pool, following the baseline is stabilized, the detection was performed by CD138 passing through at concentrations of 50 nM, 100 nM, 200 nM, 400 nM and 800 nM in sequence, with an association time of 300 s and a dissociation time of 300 s, and phosphoric acid was introduced between each concentration for regeneration.

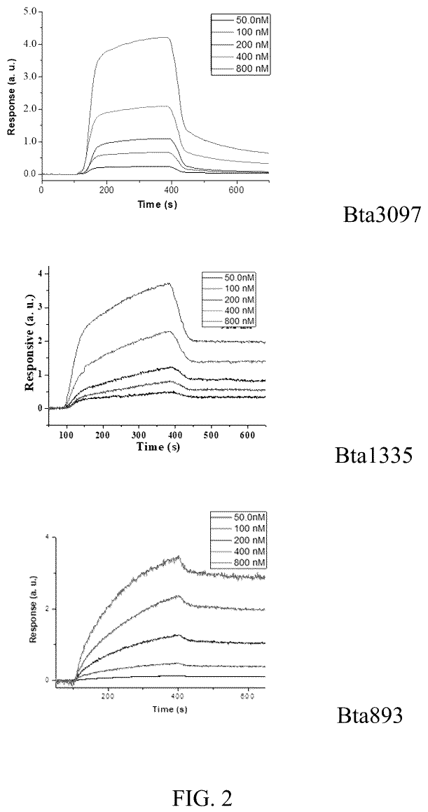

The detection results are shown in FIG. 2. The amino acid sequences of the probes obtained by high-throughput screening are:

| | Bta893: |
|---|---|
| (SEQ ID NO: 1) | |
| | His-Cys-Trp-Arg-Gly-Phe; |
| | Bta1335: |
| (SEQ ID NO: 2) | |
| | Cys-Cys-His-Arg-Gly-Phe; |
| | Bta3097: |
| (SEQ ID NO: 3) | |
| | Gly-Cys-Tyr-Arg-Arg-Phe. |

After fitted, the equilibrium dissociation constants K_{D} for polypeptide probes are 1.29×10⁻⁹ mol/L, 2.57×10⁻⁹ mol/L and 5.26×10⁻⁹ mol/L, respectively, indicating extremely high affinity.

Based on the above detection results and the analysis of the amino acid sequences of probes, the conclusions are drawn that the distinguished features of the high-affinity probes are that the N-terminus is hydrophilic and the C-terminus has a structure of Arg-AA (hydrophilic amino acid)-Phe (or other hydrophobic amino acids such as Ile); and the π-π stacking capacity of terminal groups and the length of the C-terminal chain also have an impact on the affinity of probes.

### 3. Preliminary Optimization of Probes

Based on the above conclusions, the structure of probes was further optimized. The designed probes have the features that the N-terminus is generally hydrophilic and the terminal group has π-π stacking capacity, and the C-terminus has a structure of Arg-AA (hydrophilic amino acid)-Phe (or other hydrophobic amino acids such as Ile), while the chain length of probes is controlled to regulate hydrogen bonds and hydrophobic interactions. The amino acid sequences of the designed probes are as follows:

| | BtaP1: |
|---|---|
| (SEQ ID NO: 4) | |
| | His-Cys-Trp-Arg-Arg-Phe; |
| | BtaP2: |
| (SEQ ID NO: 5) | |
| | Ile-Cys-Trp-Arg-Arg-Phe; |
| | BtaP3: |
| (SEQ ID NO: 6) | |
| | Ile-Cys-Trp-Arg-Arg-Gly-Ile. |

Synthesis of probes was performed according to the polypeptide synthesis method in Example 1.

The performance of the synthetic probes was characterized using the surface plasmon resonance imaging technology and the specific steps were as follows:
1) The synthesized probe libraries were dissolved into ddH₂O to obtain probe samples at a concentration of 100 ug/mL, respectively;
2) The probe samples were spotted on the surface of a bare gold chip, with three replicates for each sample, incubated at 4° C. for 12 hours and then the chip was immersed in 5% skimmed milk and blocked at 4° C. for 12 hours, washed with 10× PBS, 1×PBS and ultrapure water in sequence, and blow-dried by nitrogen gas;
3) The chip was mounted on the SPRi instrument, and the SPRi angle was assayed and adjusted to the optimal optical position, the relevant detection points including sample points and blank points were selected in the detection area, and the experimental flow rate was set to 2 μL/s;
4) PBS was selected as the buffer to pass into the flow pool, following the baseline is stabilized, the detection was performed by CD138 passing through at concentrations of 50 nM, 100 nM, 200 nM, 400 nM and 800 nM in sequence, with an association time of 300 s and a dissociation time of 300 s, and phosphoric acid was introduced between each concentration for regeneration.

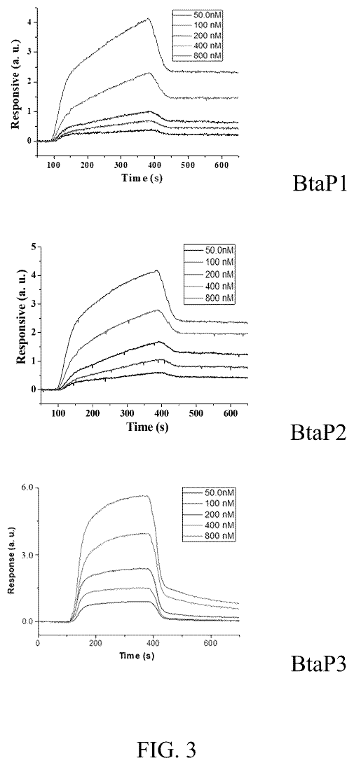

The detection results are shown in FIG. 3. After fitted, the equilibrium dissociation constants K_{D} for BtaP1-3 are 1.15×10⁻⁹ mol/L, 1.02×10⁻⁹ mol/L and 9.77×10⁻¹⁰ mol/L, respectively.

Based on the above detection results and the analysis of the amino acid sequences of probes, the conclusions are drawn that the enhanced π-π stacking of the N-terminal group and the hydrophilic residues could effectively enhance the interaction of probes with CD138, and the probes with high affinity have typical π-π stacking and intermolecular hydrogen bonds with the extracellular terminus of CD138. Meanwhile, based on the sequence of CD138, it is inferred that CD138 possesses a highly conserved sequence of EFYI (Glu-Phe-Tyr-Ile), such that the C-terminus of probes requires sufficient hydrophobicity to be embedded thereinto to form strong interactions (hydrophobic interactions and π-π stacking) with it, while all of the residues flanking the EFYI regions are hydrophilic, such that the entry of C-terminal groups of probes into hydrophobic pockets requires long hydrophilic chains to meet the requirements for hydrogen bonds and inter-pore steric hindrance. The hydrophilic chain length of probes can be further increased to provide an environment for the C-terminal group (Phe or Ile) to interact with the CD138 EFYI region and to allow the hydrophilic chain to form good intermolecular hydrogen bonds with the CD138 flanks.

### 4. Optimization of the C-Terminus of the Probes

Based on the above conclusions, the structure of probes was further optimized. The designed probes have the features that Ile-Cys-Trp structure is retained at the N-terminus and the length of the N-terminal hydrophilic chain is further extended. The amino acid sequences of the designed probes are as follows:

| | BtaPL1: |
|---|---|
| (SEQ ID NO: 7) | |
| | Ile-Cys-Trp-Arg₅-Phe; |
| | BtaPL2: |
| (SEQ ID NO: 8) | |
| | Ile-Cys-Trp-Arg₇-Phe; |
| | BtaPL3: |
| (SEQ ID NO: 9) | |
| | Ile-Cys-Trp-Arg₉-Phe; |
| | BtaPL4: |
| (SEQ ID NO: 10) | |
| | Ile-Cys-Trp-Arg₁₁-Phe; |
| | BtaPL5: |
| (SEQ ID NO: 11) | |
| | Ile-Cys-Trp-Arg₁₃-Phe. |

Synthesis of probes was performed according to the polypeptide synthesis method in Example 1.

The performance of the synthetic probes was characterized using the surface plasmon resonance imaging technology and the specific steps were as follows:
1) The synthesized probe libraries were dissolved into ddH₂O to obtain probe samples at a concentration of 100 μg/mL, respectively;
2) The probe samples were spotted on the surface of a bare gold chip, with three replicates for each sample, incubated at 4° C. for 12 hours and then the chip was immersed in 5% skimmed milk and blocked at 4° C. for 12 hours, washed with 10× PBS, 1×PBS and ultrapure water in sequence, and blow-dried by nitrogen gas;
3) The chip was mounted on the SPRi instrument, and the SPRi angle was assayed and adjusted to the optimal optical position, the relevant detection points including sample points and blank points were selected in the detection area, and the experimental flow rate was set to 2 μL/s;

4) PBS was selected as the buffer to pass into the flow pool, following the baseline is stabilized, the detection was performed by CD138 passing through at concentrations of 50 nM, 100 nM, 200 nM, 400 nM and 800 nM in sequence, with an association time of 300 s and a dissociation time of 300 s, and phosphoric acid was introduced between each concentration for regeneration.

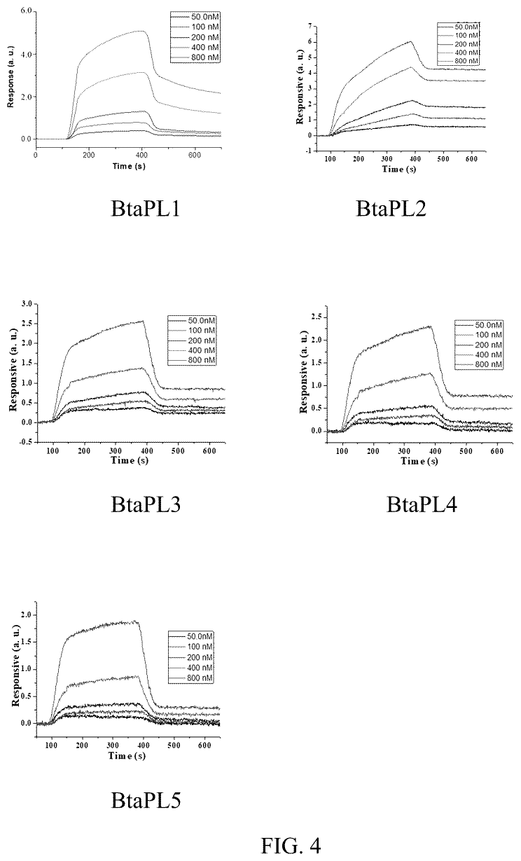

The detection results are shown in FIG. 4. After fitted, the equilibrium dissociation constants KD for BtaPL1-5 are 8.56×10⁻¹⁰ mol/L, 7.31×10⁻¹⁰ mol/L, 6.56×10⁻⁹ mol/L, 9.21×10⁻⁹ mol/L and 8.57×10⁻⁸ mol/L, respectively. The results indicate that BtaPL1-5 have very high affinity, among which the affinities of BtaPL1 and BtaPL2 are particularly prominent.

Based on the above detection results and the analysis of the amino acid sequences of probes, the conclusions are drawn that conditions that may affect the affinity of the probe include: 1. chain length, a too long chain length will cause the space at the binding interface to affect the binding at the N terminus; 2. the number of Arg repeats, the highly repeated Args enhance the intramolecular hydrogen bonds and the secondary structure of probes affects the binding of the probes to CD138.

### 5. Optimization of Intramolecular Hydrogen Bonds and Chain Length

Based on the above conclusions, the structure of probes was further optimized. The designed probes have the features that Ile-Cys-Trp structure is retained at the N-terminus, and the N-terminal hydrophilic chain avoids highly repeated Args, while the chain length is adjusted to a moderate length. The amino acid sequences of the designed probes are as follows:

| | BtaE1: |
|---|---|
| (SEQ ID NO: 12) | |
| | Ile-Cys-Trp-Arg₃Gly₃-Phe; |
| | BtaE2: |
| (SEQ ID NO: 13) | |
| | Ile-Cys-Trp-Gly₂Arg₃Gly₃-Phe; |
| | BtaE3: |
| (SEQ ID NO: 14) | |
| | Ile-Cys-Trp-Gly-Arg₃-Gly₂-Phe. |

Synthesis of probes was performed according to the polypeptide synthesis method in Example 1.

The performance of the synthetic probes was characterized using the surface plasmon resonance imaging technology and the specific steps were as follows:
1) The synthesized probe libraries were dissolved into ddH₂O to obtain probe samples at a concentration of 100 μg/mL, respectively;
2) The probe samples were spotted on the surface of a bare gold chip, with three replicates for each sample, incubated at 4° C. for 12 hours and then the chip was immersed in 5% skimmed milk and blocked at 4° C. for 12 hours, washed with 10×PBS, 1×PBS and ultrapure water in sequence, and blow-dried by nitrogen gas;
3) The chip was mounted on the SPRi instrument, and the SPRi angle was assayed and adjusted to the optimal optical position, the relevant detection points including sample points and blank points were selected in the detection area, and the experimental flow rate was set to 2 μL/s;
4) PBS was selected as the buffer to pass into the flow pool, following the baseline is stabilized, the detection was performed by CD138 passing through at concentrations of 50 nM, 100 nM, 200 nM, 400 nM and 800 nM in sequence, with an association time of 300 s and a dissociation time of 300 s, and phosphoric acid was introduced between each concentration for regeneration.

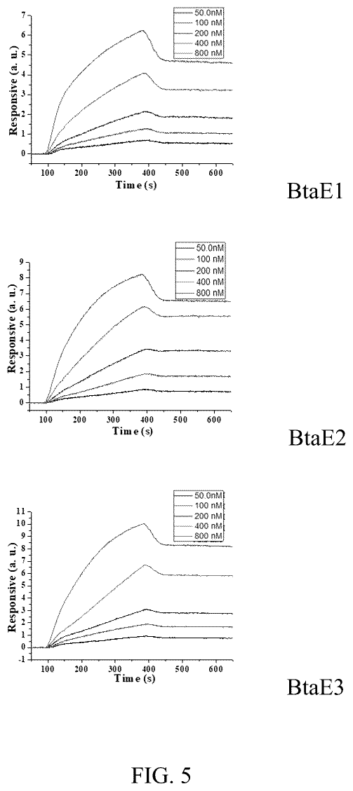

The detection results are shown in FIG. 5. After fitted, the equilibrium dissociation constants K_{D} for BtaE1-3 are 7.02×10⁻¹⁰ mol/L, 3.09×10⁻¹⁰ mol/L and 1.21×10⁻¹⁰ mol/L, respectively. The results indicate that the affinities of BtaE1-3 increase significantly, with BtaE3 having the optimal affinity results.

In the following examples, taking the BtaE3 probe as an example, the diagnostic specificity of the small molecular probe is analyzed, a functional high-throughput microarray coupled to the probe is prepared, and diagnosis of diffuse large B-cell lymphoma is performed using the probe.

### Example 2. Analysis of Specificity of the Probe

The diagnostic specificity of the small molecular probe was detected using flow cytometry and the specific steps were as follows:
1) Preparation of gradient concentration of cell samples: positive cells (diffuse large B-cell lymphoma cells) were mixed with negative cells (HELA cells) at a ratio of 1:10 and repeatedly pipetted to mix well;
2) Probe-bead coupling: the reaction was performed in PBS buffer, with a bead-coupling concentration of 1 mg/mL, and 1 mg of beads corresponded to 10 μg of the BtaE3 probe, with gentle shaking for 1 h at room temperature. This was followed by washing five times with PBS buffer containing 0.01% BSA, standing for 30 s each time, and finally resuspending in PBS buffer at a concentration of 15 mg/mL;
3) Positive cell enrichment: cells were thoroughly mixed with the magnetic bead suspension, with shaking by hand for 30 min at 4° C. in an ice box, followed by washing three times with PBS (containing 0.1% BSA and 2 mM EDTA) buffer and resuspending in PBS;
4) Specificity analysis: The captured cells were separated using a magnetic field, and the protein expression profile was quantified by flow cytometry to calculate the proportion of positive cells.

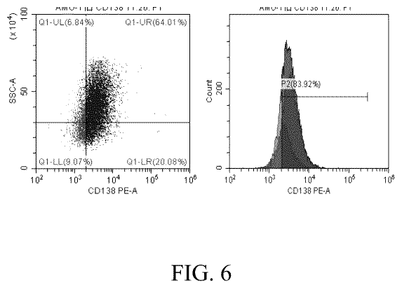

FIG. 6 shows the characterization results of flow cytometry, which indicates >90% specificity for detection using the probe of the present disclosure.

### Example 3: Preparation of Probe Array Chips

Functional high-throughput microarray chips coupled with small molecular probes were prepared by G2.5 Sputter, PECVD, photoetching and other processes and the specific steps were as follows:
1. Microarray patterning: the glass was subjected to standard pre-cleaning, followed by being spin-coated with a layer of OC photoresist (adhesion layer), followed by deposition of PVX layer (SiO₂ 1000 A+SiNx 2000 A), photoetching, protecting and patterning by coating photoresist, Coating 30Kpa\300 rpm*10 s, Pre bake 90° C. for 120 s; exposing after two repetitions, developing for 100 s, and Hard Bake 230° C. for 30 min, followed by ICP etching;
2. Construction of a bare gold array: 240° C. Au 300 nm coating, RIE etch;
3. The screened probes were coupled to the surface of the array.

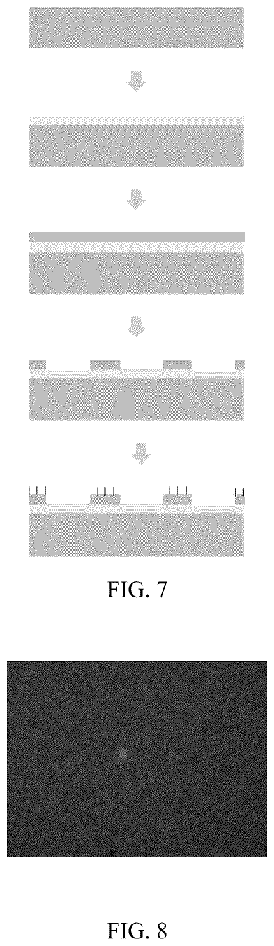

FIG. 7 is a schematic diagram of the technological process of chip preparation.

### Example 4. Detection of Diffuse Large B-Cell Lymphoma

Diffuse large B-cell lymphoma was detected using probes screened and prepared by methods in the above examples, and the specific methods were as follows:
1. Sample collection: Blood samples were collected and stored at room temperature. Blood was collected using disposable, anticoagulated, vacuum blood tubes. Generally, EDTA or heparin was used for anticoagulation; and the volume of blood collected was typically 2-5 mL.
2. Sample processing: 2 mL of lymphocyte separation solution was added into a sterile plastic centrifuge tube, and 2 mL of PBS buffer was added into a vacuum tube containing 2 mL of the blood sample and mixed in 1:1. The mixed blood sample-PBS mixture was slowly added into the centrifuge tube with lymphocyte separation solution, note that it should be slowly, allowing the blood sample located in the upper layer of the extract as much as possible, and then centrifuged at 1500 rpm at 20° C. for 15 min. After centrifugation, 4 layers were present in the tube, which, from top to bottom, were layers of plasma, mononuclear cells, granular leukocytes and erythrocytes. A capillary tube was extended into the mononuclear cell layer (located at the interface between the cell separation solution and the plasma) and all cells were gently aspirated along the tube wall, which was then washed twice with Hanks' solution with centrifugation at 12000 r/min for 10 min each time.
3. Probe immobilization: Small molecular probe coupling was performed on the surface of glass chips coated with bare gold. The polypeptide probes were dissolved with pure water to prepare a probe solution at concentration of 1 mM, and the probe solution was applied to the chip surface and incubated at 4° C., 40% humidity for 24 hours, followed by washing with 10×PBS, 1×PBS and ultrapure water in sequence, respectively, and blow-dry by nitrogen gas.
4. Probe capture of tumor cells: The lymphocytes obtained by extraction were mixed into 1X PBST and dispersed to ensure that no agglomeration occurred, and then the cell suspension was diluted with 1×PBST to 1*10⁵/mL and passed through the surface of the chip coupled with the probes at a flow rate of 5 μL/s for 500 s, followed by passing through the surface of the chip with 1×PBST at a flow rate of 3 μL/s for 500 s to fully dissociate the non-specific adsorption;
5. Characterization of cells by staining: The captured cells were bound by using Rhodamine B-labeled anti-CD138 antibody (at a site different from the antibody used for capture), characterized by fluorescence microscopy, and the captured positive cells were verified, thereby allowing the diagnosis of diffuse large B-cell lymphoma.

FIG. 8 shows the results of the detection of diffuse large B-cell lymphoma using the polypeptide probe of the present disclosure. The results indicate that the probe of the present disclosure can specifically and efficiently detect diffuse large B-cell lymphoma cells and thus can be used for the diagnosis of diffuse large B-cell lymphoma.

The applicant declares that the present invention illustrates the process method of the present invention by means of the above mentioned examples, but the present invention is not limited to the above mentioned process steps, that is, it does not mean that the present invention must rely on the above mentioned process steps in order to be implemented. It should be obvious to one skilled in the art that any improvement of the present invention, equivalent substitution of the raw materials selected for the present invention and the addition of auxiliary ingredients, the choice of specific embodiments and the like fall within the scope of protection and disclosure of the present invention.

This application incorporates by reference a Sequence Listing with this application as an ASCII text file named “C22W2128.01US—sequence listing” created on Aug. 10, 2024, and having a size of 2,833 bytes.

1. A probe specifically binding to CD138 having, from the N-terminus to the C-terminus, the following structure:
X-M-Arg-Y-Phe or X-M-Arg-Y-Ile,
wherein X is any amino acid residue, M is any amino acid residue or absent, and the number of amino acid residues represented by X+M ranges from 3-15, and the N-terminal amino acid residues or the amino acid residues represented by X+M are overall hydrophilic, and
wherein Y is selected from one or more of the following amino acid residues: Arg, Gly, Tyr, Asn, Gln, Ser, Thr, Cys, and Sec, and the number of amino acid residues represented by Y ranges from 1-12.

2. The probe of claim 1, wherein the N-terminal amino acid residues or amino acid residues represented by X+M, and/or the C-terminal amino acid residues or amino acid residues represented by Arg-Y-Phe or Arg-Y-Ile have a x-x stacking capability.

3. The probe of claim 1, wherein the probe is selected from the group consisting of:

| | Bta893: |
|---|---|
| (SEQ ID NO: 1) | |
| | His-Cys-Trp-Arg-Gly-Phe; |
| | Bta1335: |
| (SEQ ID NO: 2) | |
| | Cys-Cys-His-Arg-Gly-Phe; |
| | Bta3097: |
| (SEQ ID NO: 3) | |
| | Gly-Cys-Tyr-Arg-Arg-Phe; |
| | BtaP1: |
| (SEQ ID NO: 4) | |
| | His-Cys-Trp-Arg-Arg-Phe; |
| | BtaP2: |
| (SEQ ID NO: 5) | |
| | Ile-Cys-Trp-Arg-Arg-Phe; |
| | and |
| | BtaP3: |
| (SEQ ID NO: 6) | |
| | Ile-Cys-Trp-Arg-Arg-Gly-Ile. |

4. The probe of claim 1, wherein the X is Ile-Cys-Trp.

5. The probe of claim 4, wherein the probe is selected from the group consisting of:

| | BtaPL1: |
|---|---|
| (SEQ ID NO: 7) | |
| | Ile-Cys-Trp-Arg₅-Phe; |
| | BtaPL2: |
| (SEQ ID NO: 8) | |
| | Ile-Cys-Trp-Arg₇-Phe; |
| | BtaPL3: |
| (SEQ ID NO: 9) | |
| | Ile-Cys-Trp-Arg₉-Phe; |
| | BtaPL4: |
| (SEQ ID NO: 10) | |
| | Ile-Cys-Trp-Arg₁₁-Phe; |
| | and |
| | BtaPL5: |
| (SEQ ID NO: 11) | |
| | Ile-Cys-Trp-Arg₁₃-Phe. |

6. The probe of claim 4, wherein the Y is selected from one or both of the following amino acid residues: Arg and Gly.

7. The probe of claim 6, wherein the probe is selected from the group consisting of:

| | BtaE1: |
|---|---|
| (SEQ ID NO: 12) | |
| | Ile-Cys-Trp-Arg₃Gly₃-Phe; |
| | BtaE2: |
| (SEQ ID NO: 13) | |
| | Ile-Cys-Trp-Gly₂Arg₃Gly₃-Phe; |
| | and |
| | BtaE3: |
| (SEQ ID NO: 14) | |
| | Ile-Cys-Trp-Gly-Arg₃-Gly₂-Phe. |

8. A kit comprising the probe of any one of claim 1.

9. The kit of claim 8, wherein the probe is labeled by fluorescein or biotin.

10. The kit of claim 8, further comprising a surfactant, a buffer and EDTA.

11. A microfluidic chip comprising the probe of claim 1 immobilized on a solid substrate.

12. A method of screening a probe for diagnosing diffuse large B-cell lymphoma, comprising the steps of:
i) constructing a probe library,
ii) enriching the probe by using CD138,
iii) optimizing the probe for the following features:
a) the probe having, from the N-terminus to the C-terminus, the following structure: X-M-Arg-Y-Phe or X-M-Arg-Y-Ile, wherein X is any amino acid residue, M is any amino acid residue or absent, and the number of N-terminal amino acid residues or represented by X+M ranges from 3-15,
b) the N-terminal amino acid residues or the amino acid residues represented by X+M being overall hydrophilic,
c) Y being selected from one or more of the following amino acid residues: Arg, Gly, Tyr, Asn, Gln, Ser, Thr, Cys, and Sec, and the number of amino acid residues represented by Y ranging from 1-12, and
d) the probe having a length ranging from 5-18 amino acids.

13. The method of claim 12, further comprising optimizing the probe for the feature selected from:
e) the N-terminal amino acid residues or amino acid residues represented by X+M, and/or the C-terminal amino acid residues or amino acid residues represented by Arg-Y-Phe or Arg-Y-Ile having a π-π stacking ability,
f) the X being Ile-Cys-Trp, and
g) the Y being selected from one or both of the following amino acid residues: Arg and Gly.

14. A method of diagnosing diffuse large B-cell lymphoma in a subject, comprising
a) obtaining a blood sample from the subject,
b) obtaining a mononuclear cell fraction from the blood sample,
c) contacting the probe of claim 1 with the mononuclear cell fraction, and
d) detecting whether lymphoma cells are captured using visualization means, thereby diagnosing whether the subject suffers from diffuse large B-cell lymphoma.

15. (canceled)

16. The probe of claim 1, wherein the probe has a length ranging from 5-18 amino acids.

17. The kit of claim 8, wherein the probe is labeled by Rhodamine B.

18. The kit of claim 10, wherein the surfactant is selected from one or a combination of Tween 20, sodium dodecyl sulfate (SDS) and sorbitan fatty acid esters (Span), and the buffer is PBS.

19. The microfluidic chip of claim 11, wherein the probe is coupled to the surface of the solid substrate, and the solid substrate is glass.

20. A method of diagnosing diffuse large B-cell lymphoma in a subject, comprising
a) obtaining a blood sample from the subject,
b) obtaining a mononuclear cell fraction from the blood sample,
c) contacting the kit of claim 8 with the mononuclear cell fraction, and
d) detecting whether lymphoma cells are captured using visualization means, thereby diagnosing whether the subject suffers from diffuse large B-cell lymphoma.

21. A method of diagnosing diffuse large B-cell lymphoma in a subject, comprising
a) obtaining a blood sample from the subject,
b) obtaining a mononuclear cell fraction from the blood sample,
c) contacting the microfluidic chip of claim 11 with the mononuclear cell fraction, and
d) detecting whether lymphoma cells are captured using visualization means, thereby diagnosing whether the subject suffers from diffuse large B-cell lymphoma.


SEQUENCE LISTING (14 sequences)

SEQ ID NO: 1 - protein, 6 aa, Artificial Sequence
  Bta893
HCWRGF

SEQ ID NO: 2 - protein, 6 aa, Artificial Sequence
  Bta1335
CCHRGF

SEQ ID NO: 3 - protein, 6 aa, Artificial Sequence
  Bta3097
GCYRRF

SEQ ID NO: 4 - protein, 6 aa, Artificial Sequence
  BtaP1
HCWRRF

SEQ ID NO: 5 - protein, 6 aa, Artificial Sequence
  BtaP2
ICWRRF

SEQ ID NO: 6 - protein, 7 aa, Artificial Sequence
  BtaP3
ICWRRGI

SEQ ID NO: 7 - protein, 9 aa, Artificial Sequence
  BtaPL1
ICWRRRRRF

SEQ ID NO: 8 - protein, 11 aa, Artificial Sequence
  BtaPL2
ICWRRRRRRRF

SEQ ID NO: 9 - protein, 13 aa, Artificial Sequence
  BtaPL3
ICWRRRRRRRRRF

SEQ ID NO: 10 - protein, 15 aa, Artificial Sequence
  BtaPL4
ICWRRRRRRRRRRRF

SEQ ID NO: 11 - protein, 17 aa, Artificial Sequence
  BtaPL5
ICWRRRRRRRRRRRRRF

SEQ ID NO: 12 - protein, 10 aa, Artificial Sequence
  BtaE1
ICWRRRGGGF

SEQ ID NO: 13 - protein, 12 aa, Artificial Sequence
  BtaE2
ICWGGRRRGGGF

SEQ ID NO: 14 - protein, 10 aa, Artificial Sequence
  BtaE3
ICWGRRRGGF